(19) United States
(12) Patent Application Publication
(10) Pub. No.: US 2018/0061097 A1
(43) Pub. Date: Mar. 1, 2018

(54) IMAGE GENERATION APPARATUS, IMAGE GENERATION METHOD, AND X-RAY CT APPARATUS

(71) Applicant: HITACHI, LTD., Tokyo (JP)

(72) Inventors: Kazuma YOKOI, Tokyo (JP); Yushi TSUBOTA, Tokyo (JP)

(21) Appl. No.: 15/561,231

(22) PCT Filed: Mar. 8, 2016
(86) PCT No.: PCT/JP2016/057108

§ 371 Date: Sep. 25, 2017

(30) Foreign Application Priority Data
Mar. 30, 2015 (JP) 2015-068965

(51) Int. Cl. G06T 11/20; A61B 6/03; G06T 11/00

(52) U.S. Cl. CPC G06T 11/206; G06T 11/005; A61B 6/032

(57) ABSTRACT

The purpose of the present invention is to reduce statistical errors in material decomposition images. In order to achieve this purpose, the present invention is characterized by having: a scatter plot generation unit (414) that generates a scatter plot in which the axes represent the concentrations of base materials used in base material decomposition and pixels of a material decomposition image output as a result of the base material decomposition are plotted against the corresponding concentrations of the base materials of the base material decomposition; an angle processing unit (415) that rotates the scatter plot to minimize the statistical errors of plot points plotted thereon; and a pixel conversion unit (416) that converts the pixels of the material decomposition image on the basis of the pixels in the scatter plot where the statistical errors are minimized by the error minimization unit.

### TECHNICAL FIELD

The present invention relates to technologies about image generation apparatuses that correct material decomposition images, image generation methods, and X-ray CT apparatuses.

### BACKGROUND ART

Typically, an X-ray CT (Computerized Tomography) apparatus has a configuration in which an X-ray photon group that has a continuous (nonmonochromatic) energy distribution and is emitted from an X-ray tube is detected by an X-ray detector that operates in a current mode. However, there is a problem in that an X-ray detector that operates in a current mode cannot acquire energy information.

Roughly speaking, as technologies for effectively utilizing information brought about by an X-ray group having plural energy distributions, there are two methods. One is a dual energy CT, and it operates in a current mode as a detector without change, and uses a technique in which two continuous energy distributions brought about by two kinds of X-ray tube voltages are used. The other is a technique called a photon counting CT, a spectral CT, or the like, and it is a technique in which a pulse mode detector, which can acquire energy information, is used.

In addition, a technology in which weighted addition is executed in order to reduce statistical errors is disclosed (for example, disclosed in Patent Literature 1).

### CITATION LIST

### Patent Literature

Patent Literature 1: Japanese Unexamined Patent Application Publication No. 2006-101926

### SUMMARY OF INVENTION

### Technical Problem

Although an X-ray CT apparatus using a pulse mode detector can acquire information which an X-ray CT apparatus that operates in a current mode cannot acquire, the statistical errors of a material decomposition image obtained by the X-ray CT apparatus using a pulse mode detector is usually not excellent. If the statistical errors are not excellent, the material decomposition image is blurred. Therefore, an image with small statistical errors is desired in order to secure fundamental visibility and to separate regions of interest from each other.

Furthermore, a technology disclosed in Patent Literature 1 needs a high calculation cost as well and does not necessarily minimize errors .

The present invention was achieved with such a background in mind, and a problem to be solved by the present invention is to reduce statistical errors in a material decomposition image.

### Solution to Problem

In order to solve the abovementioned problem, the present invention is characterized by including: a scatter plot generation unit that generates a scatter plot in which the axes represent the concentrations of base materials used in base material decomposition and pixels of a material decomposition image output as a result of the base material decomposition are plotted against the corresponding concentrations of the base materials of the material decomposition; an error minimizing unit that rotates the scatter plot in a direction that minimizes the statistical errors of plot points plotted on the scatter plot; and a conversion unit that converts the material decomposition image on the basis of the pixels in the scatter plot rotated by the error minimizing unit.

Other solutions will be described in the following embodiment.

### Advantageous Effects of Invention

According to the present invention, statistical errors in a material decomposition image can be made small.

### BRIEF DESCRIPTION OF DRAWINGS

FIG. 1 is a schematic configuration diagram of an X-ray CT apparatus as a target of this embodiment.

FIG. 2 is a functional block diagram showing the configuration of an image generation apparatus according to this embodiment.

FIG. 3 is a flowchart showing the procedure of an error minimized image generation processing according to this embodiment.

FIG. 4 is a diagram showing an example of a scatter plot of a material decomposition image.

FIG. 5 is a diagram showing examples of HAp image histograms.

FIG. 6 is a diagram showing an example of a scatter plot after rotation processing.

FIG. 7 is a diagram showing an example of a HAp image histogram generated on the basis of a rotated scatter plot.

FIG. 8 is a flowchart showing the procedure of pixel conversion processing according to this embodiment.

FIG. 9 is a diagram showing an example of an operation screen according to this embodiment.

### DESCRIPTION OF EMBODIMENT

Next, a configuration for implementing the present invention (referred to as an “embodiment” hereinafter) will be explained in detail by accordingly referring to the accompanying drawings.

### [X-Ray CT Apparatus]

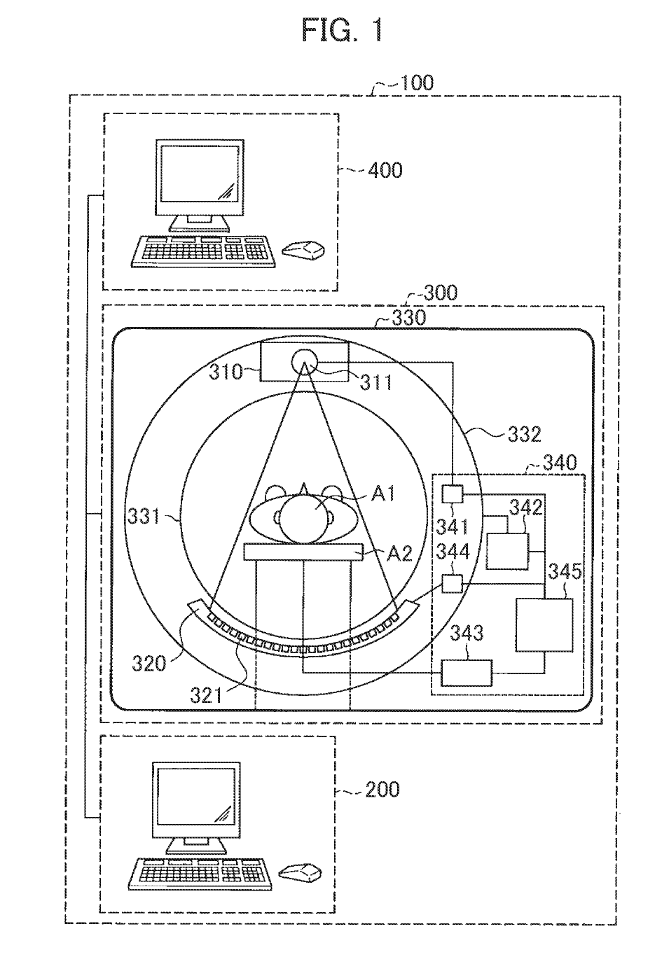

FIG. 1 is a schematic configuration diagram of an X-ray CT apparatus regarding as a target of this embodiment.

The X-ray CT apparatus 100 includes an input apparatus 200, a photographing apparatus 300, and an image generation apparatus 400.

In addition, the photographing apparatus 300 includes an X-ray generation device 310, an X-ray detection device 320, a gantry 330, a photographing control device 340, and a test substance mounting table A2.

The input apparatus 200 is used for inputting information to control the photographing apparatus 300. The image generation apparatus 400 is an apparatus that acquires count projection data photographed by the photographing apparatus 300, and performs image processing on the count projection data.

Here, it is not always necessary that the input apparatus 200 and the image generation apparatus 400 are provided separately from the X-ray CT apparatus 100, and they can be provided in an all-in-one configuration.

Alternatively, an apparatus having both functions of the image generation apparatus 400 and the input apparatus 200 can be used to achieve the abovementioned processing.

The X-ray generation device 310 in the photographing apparatus 300 includes an X-ray tube 311. Furthermore, the X-ray detection device 320 includes an X-ray detector 321. Here, it will be assumed in this embodiment that the X-ray detector 321 is a pulse mode X-ray detector.

In addition, a circular opening 331 for disposing a test substance A1 and the test substance mounting table A2 is installed in the center of the gantry 330. A rotary table 332 for mounting the X-ray tube 311 and the X-ray detector 321, and a driving mechanism (not shown) for rotating the rotary table 332 are installed inside the gantry 330.

Furthermore, a driving mechanism (not shown) for adjusting the position of the test substance A1 relative to the gantry 330 is installed on the test substance mounting table A2.

Furthermore, the photographing control device 340 includes: an X-ray control circuit 341 for controlling the X-ray tube 311; a gantry control circuit 342 for controlling the rotary drive of the rotary table 332; a table control circuit 343 for controlling the drive of the test substance mounting table A2; a detector control circuit 344 for controlling the photographing of the X-ray detector 321; and an overall control circuit 345 for controlling the flow of the operations of the X-ray control circuit 341, the gantry control circuit 342, the table control circuit 343, and the detector control circuit 344.

### (X-Ray Tube, X-Ray Detector, Photographing Apparatus)

A distance between the X-ray emission point of the X-ray tube 311 and the X-ray incoming plane of the X-ray detector 321 is, for example, 1000 mm. The diameter of the opening 331 of the gantry 330 is, for example, 700 mm. As the X-ray detector 321, a publicly known X-ray detector including a scintillator (an element that emits fluorescence on receiving X-ray or ionizing radiation) and a photodiode (an element that converts light such as fluorescence into electricity) is used. The X-ray detector 321 has a configuration including a large number of detection elements that are arranged in a circular arc shape so as to be equally displaced from the X-ray emission point of the X-ray tube 311, and the number of the detection elements (the number of channels) is, for example, 1000. The size of each detection element in the direction of its channel is, for example, 1 mm.

As the X-ray detector 321, not only a semiconductor X-ray detector including a scintillator and a photodiode, but also a semiconductor X-ray detector including a CdTe (cadmium telluride) can be used.

A time required for the rotation of the rotary table 332 depends on parameters input by a user using the input apparatus 200. The time required for the rotation is, for example, 1.0 s/rotation.

The number of photographings per rotation of the photographing apparatus 300 is, for example, 900. In other words, one photographing is executed every 0.4-degree rotation of the rotary table 332.

Here, the values of the above specifications are not limited to these values, and can be changed variously in accordance with the configuration of the X-ray CT apparatus 100.

### [Image Generation Apparatus]

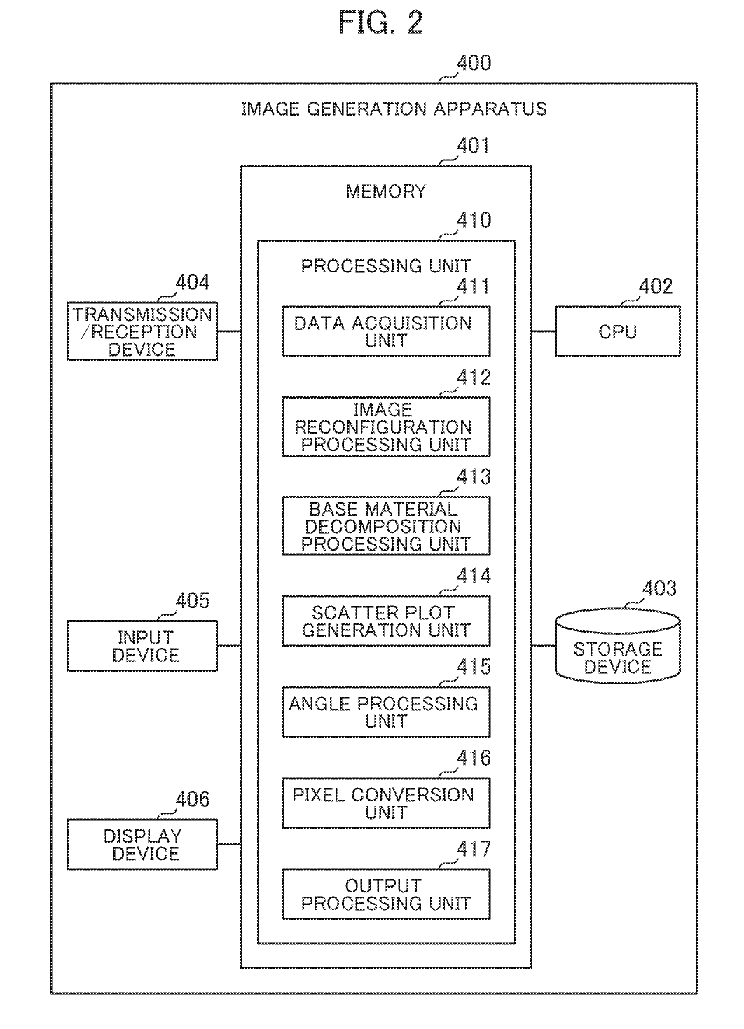

FIG. 2 is a functional block diagram showing the configuration of the image generation apparatus according to this embodiment.

The image generation apparatus 400 includes: a memory 401; a CPU (Central Processing Unit) 402; a storage device 403 such as a HD (Hard Disc); a transmission/reception device 404; an input device 405; and a display device 406.

Programs stored in the storage device 403 are expanded into the memory 401, and the expanded programs are executed by the CPU 402, which makes it possible to realize a processing unit 410, and units included in the processing unit 410, that is to say, a data acquisition unit 411, an image reconfiguration processing unit 412, a base material decomposition processing unit 413, a scatter plot generation unit 414, an angle processing unit (an error minimizing unit) 415, a pixel conversion unit (a conversion unit) 416, and an output processing unit 417. In addition, the details of processing performed by the individual units 411 to 417 will be explained later.

The data acquisition unit 411 acquires count projection data from the photographing apparatus 300.

The image reconfiguration processing unit 412 generates a line attenuation coefficient image on the basis of the acquired count projection data.

The base material decomposition processing unit 413 performs basic material decomposition processing using a base material line attenuation coefficient and the line attenuation coefficient image.

The scatter plot generation unit 414 plots the pixels of a material decomposition image obtained as a result of the base material decomposition processing on a scatter plot whose axes are represented by information about the base materials.

The angle processing unit 415 calculates a rotation angle on the basis of the generated scatter plot. The rotation angle will be explained later. Furthermore, the angle processing unit 415 rotates the scatter plot on the basis of the calculated rotation angle.

By converting the pixels of the material decomposition image on the basis of the rotated scatter plot, the pixel conversion unit 416 generates an error minimized image (an image after being converted). The error minimized image will be explained later.

The output processing unit 417 displays the processing results of the respective units 411 to 416 on the display device 406.

The transmission/reception device 404 receives count projection data and the like from the photographing apparatus 300 (shown in FIG. 1), and transmits the count projection data and the like to the data acquisition unit 411.

The input device 405 is a keyboard, a mouse, or the like, and information about the rotation of a scatter plot, coordinate conversion, or the like is input.

The display device (a display unit) 406 is a display or the like, and displays the results of the respective processing.

### [Flowchart]

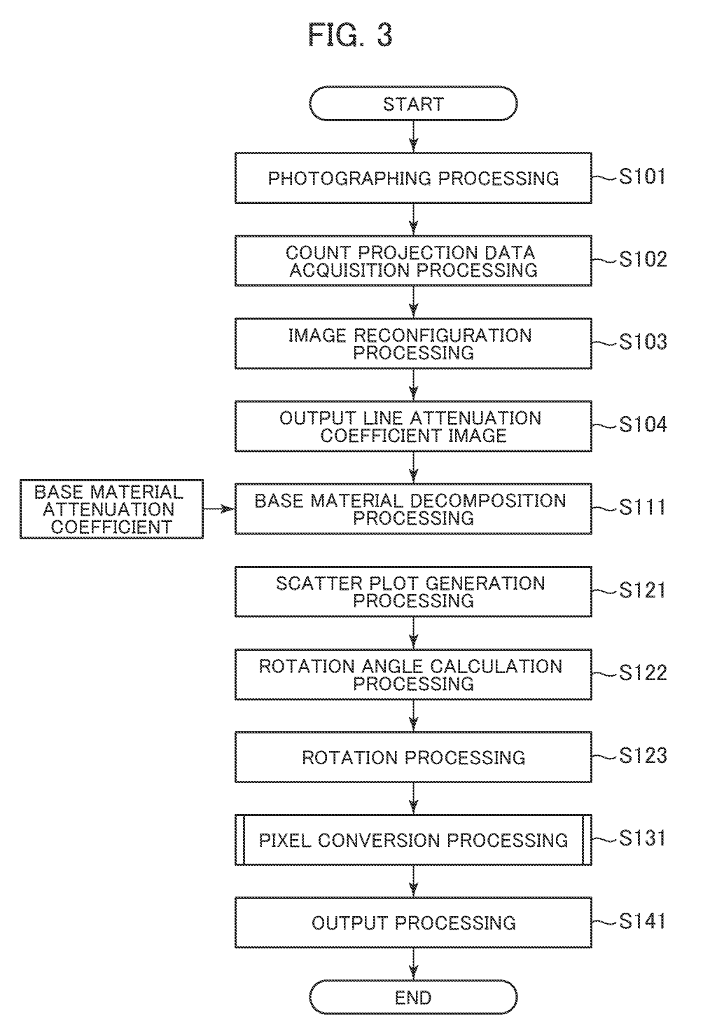

FIG. 3 is a flowchart showing the procedure of an error minimized image generation processing according to this embodiment. The flowchart shown in FIG. 3 will be explained with reference to FIG. 1 and FIG. 2 accordingly. Here, this application is characterized by processing shown in step S121 to step S141.

First, the photographing apparatus 300 performs photographing processing for photograph the test substance A1 (S101).

Next, the data acquisition unit 411 performs count projection data acquisition processing in which count projection data is acquired for every energy window from the photographing apparatus 300 (S102).

An energy window division number N is limited by the mounting density of a circuit for realizing a pulse mode X-ray detector, the upper limit of heat generation of the circuit, a data transfer rate, and the like, and it is preferable that the division number N is about 3 to 8.

Subsequently, the image reconfiguration processing unit 412 performs image reconfiguration processing on the acquired count projection data N times for every energy window (S103).

As a result, one line attenuation coefficient image is output for every energy window (S104). Here, because the count projection data is distributed to N energy windows (N is the number of energy windows), the statistical errors of a line attenuation coefficient image for each energy window obtained by the X-ray CT apparatus 100 according to this embodiment become larger than those obtained by an X-ray CT apparatus that operates in a current mode.

If the atomic composition and mass density of a certain material are known, a line attenuation coefficient of the material for each energy is uniquely determined. However, because each energy window has a width, a beam hardening effect occurs in a line attenuation coefficient image for each energy window and the material size of a test substance A1 affects actually-measured line attenuation coefficients. In this case, it will be assumed that it is possible that, by acquiring in advance actually-measured line attenuation coefficients obtained from the reconfiguration of phantoms with plural sizes and shapes, beam hardening correction is executed so that the beam hardening effect becomes substantially small. Here, the phantoms are evaluation appliances for calibration used for regular checkups or daily checkups using medical diagnostic imaging apparatuses such as an X-ray CT apparatus 100 and an MRI (magnetic resonance imaging) apparatus. In addition, the reconfiguration of phantoms means that the phantoms are mounted on an X-ray CT apparatus 100 for correcting the absorption coefficient of a material and the like, and then photographing is executed, and image reconfiguration processing is performed.

Next, M kinds of base materials are set for executing base material decomposition. The base materials are selected by a user as his/her favorite materials of interest corresponding to the relevant examination. In the case of M=2, although typical base materials are water and iodine, fat and hydroxyapatite (referred to as HAp hereinafter) for the observation of arteriosclerosis are used. In addition, although there are many cases where an origin is vacuum (nearly equal to air), it is assumed that the origin is blood in this case. Here, the origin is the origin of a scatter plot that will be described later.

The base material decomposition processing unit 413 performs base material decomposition processing using base material line attenuation coefficients corresponding to the set base materials and the line attenuation coefficient images output at step S104 (S111). Hereby, a material decomposition image is generated.

Furthermore, in the case where appropriate beam hardening correction can be executed, the base material line attenuation coefficients are uniquely determined corresponding to the set base material group, thereby they can be treated as known values.

If the number of the base materials (base material number) M is equal to the number of energy windows N or smaller, the base material decomposition has a solution or a least squares solution, and a material decomposition image is output. However, because the line attenuation coefficient images have statistical errors, a combination of base materials that are substantially decomposable is limited to a combination of base materials whose atomic numbers are far apart from each other. Hereinafter, descriptions will be made under the assumption that an appropriate material decomposition image can be obtained.

Next, the scatter plot generation unit 414 performs scatter plot generation processing in which the scatter plot of the material decomposition image generated at step S111 is generated (S121). The scatter plot of the material decomposition image will be described later.

Subsequently, the angle processing unit 415 calculates an angle in the longitudinal direction of a homogeneous region in the scatter plot of the material decomposition image, and on the basis of this angle, the angle processing unit 415 performs rotation angle calculation processing for calculating a rotation angle (S122). The processing at step S122 will be described later.

Subsequently, the angle processing unit 415 performs rotation processing in which the scatter plot is rotated in accordance with the calculated rotation angle (S123).

In addition, the pixel conversion unit 416 performs pixel conversion processing in which the pixels of the material decomposition image are replaced with the pixels of the rotated scatter plot (S131). The pixel conversion processing will be described later.

Successively, the output processing unit 417 performs output processing in which the processing result of step S123 and the processing result of the step S131 are output to the display device 406 (S141).

### (Scatter Plot)

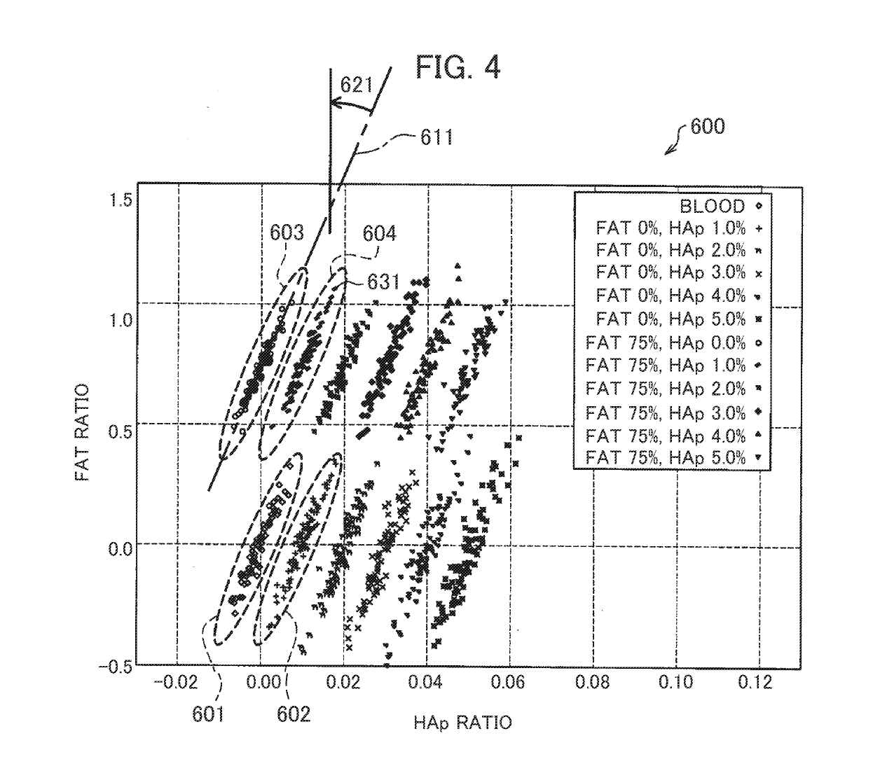

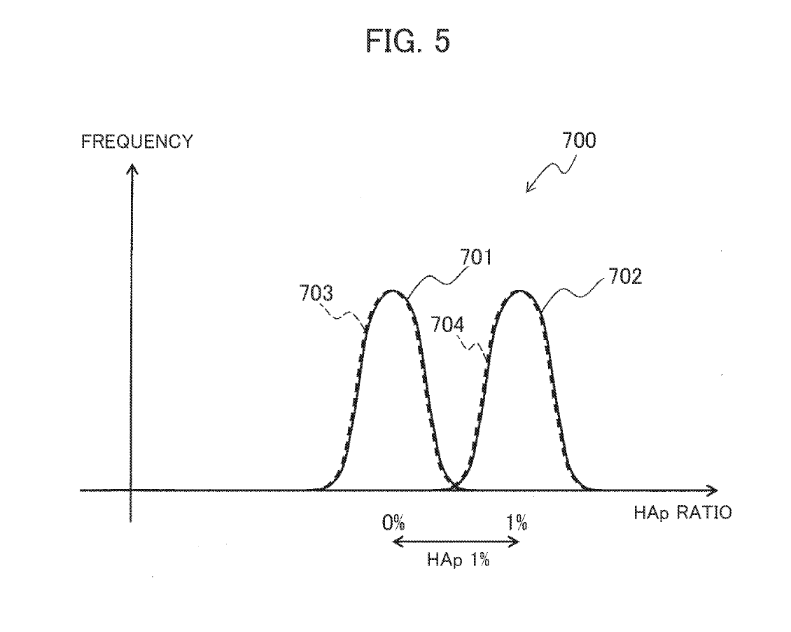

FIG. 4 is a diagram showing an example of a scatter plot of a material decomposition image, and FIG. 5 is a diagram showing examples of HAp image histograms.

The scatter plot 600 shown in FIG. 4 is a scatter plot generated at step S121 shown in FIG. 3.

The scatter plot 600 is a scatter plot using both HAp image and fat image of the material decomposition image. Here, the material decomposition image is an image generated at step S111 shown in FIG. 3.

In the scatter plot 600, the horizontal axis represents a HAp ratio (a base material concentration), and the vertical axis represents a fat ratio (a base material concentration). The vertical axis is referred to as a fat ratio axis, and the horizontal axis is referred to as a HAp ratio axis accordingly. Each plot point in the scatter plot 600 corresponds to each pixel of the material decomposition image, and this scatter plot shows to which HAp ratio (which base material concentration) and to which fat ratio (which base material concentration) each pixel of the material decomposition image corresponds. The material ratio of the HAp ratio, the material ratio of the fat ratio, and the like are the volume ratios of the base materials (HAp and fat in this case) respectively relative to the space.

Furthermore, the scatter plot 600 shows six kinds of HAp ratios (0 to 5%) and two kinds of fat ratios (0% and 75%), in which reference signs 601 to 604 are included as examples.

HAp image histograms 700 shown in FIG. 5 are histograms obtained by projecting each plot point of the scatter plot 600 shown in FIG. 4 onto the HAp ratio axis. In other words, the vertical axis in the HAp image histograms 700 in FIG. 5 represents numbers obtained by counting the numbers of plot points of FIG. 4 in the direction of the vertical axis of FIG. 4. The horizontal axis of FIG. 5 is the same as that of FIG. 4.

To put it concretely, under the assumption that homogeneous regions including reference signs 601 and 602 are regions of interest (ROIs) in the scatter plot 600 shown in FIG. 4, the HAp image histograms 700 shown in FIG. 5 are histograms regarding the ROIs. The ROIs are regions in which the material ratios (the HAp ratio and the fat ratio) are considered to be homogeneous (in other words, an attenuation coefficient in each energy window is homogeneous), and the ROIs are obtained from the pixel values of the material decomposition image. For example, from the fact that an attenuation coefficient in each energy window is within a narrow certain range and the like, the scatter plot generation unit 414 judges which regions are the ROIs.

In FIG. 5, a solid line represented by a reference sign 701 shows a histogram showing an error distribution of a region corresponding to fat 0% & HAp 0% (blood) (corresponding to the homogeneous region 601 in FIG. 4), and a solid line represented by a reference sign 702 shows a histogram showing an error distribution of a region corresponding to fat 0% & HAp 1% (corresponding to the homogeneous region 602 in FIG. 4). Furthermore, a dashed line represented by a reference sign 703 shows a histogram showing an error distribution of a region corresponding to fat 75% & HAp 0% (corresponding to the homogeneous region 603 in FIG. 4), and a dashed line represented by a reference sign 704 shows a histogram showing an error distribution of a region corresponding to fat 75% & HAp 1% (corresponding to the homogeneous region 604 in FIG. 4). The histogram 701 and the histogram 703 overlap each other, and the histogram 702 and the histogram 704 overlap each other. It will be assumed that materials are homogeneous in the region of each histogram, and a material other than fat and HAp is blood. Judging from FIG. 5, it is found that the average values (central values) of the homogeneous regions 601 and 602 based on the HAp image of the material decomposition image depend only on the HAp ratios and are plotted independently of the fat ratios. In other words, as mentioned above, the histogram 701 and the histogram 703 overlap each other, and the histogram 702 and the histogram 704 overlap each other. This is an advantageous characteristic from the viewpoint of diagnosis. However, the image quality of the material decomposition image based on FIG. 4 or FIG. 5 is not found excellent, and the material decomposition image has a tendency to have large statistical errors. In other words, each of the histograms 701 to 704 has a wide width. As mentioned above, if the respective histograms 701 to 704 have wide widths, the material decomposition image has a vague image.

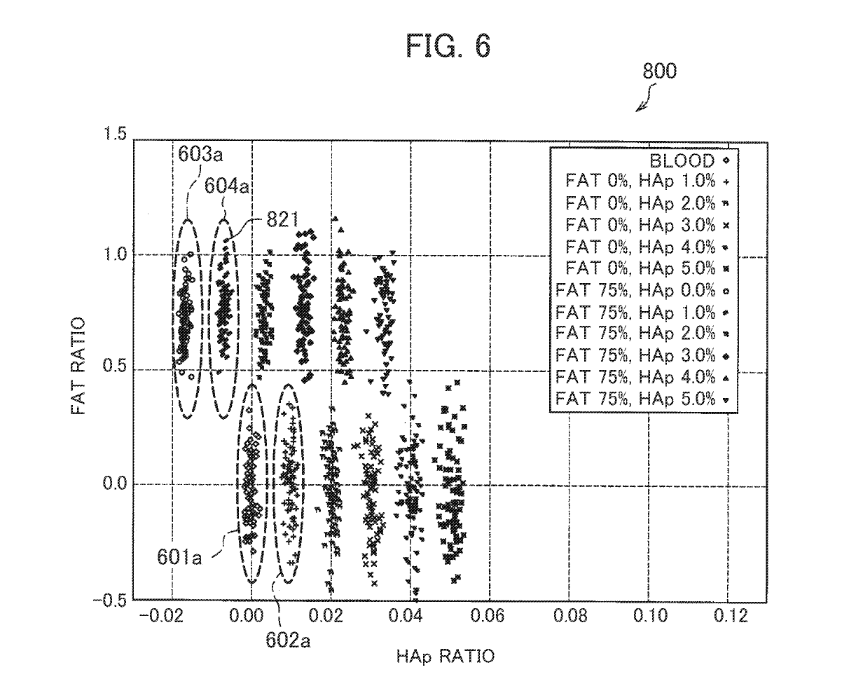

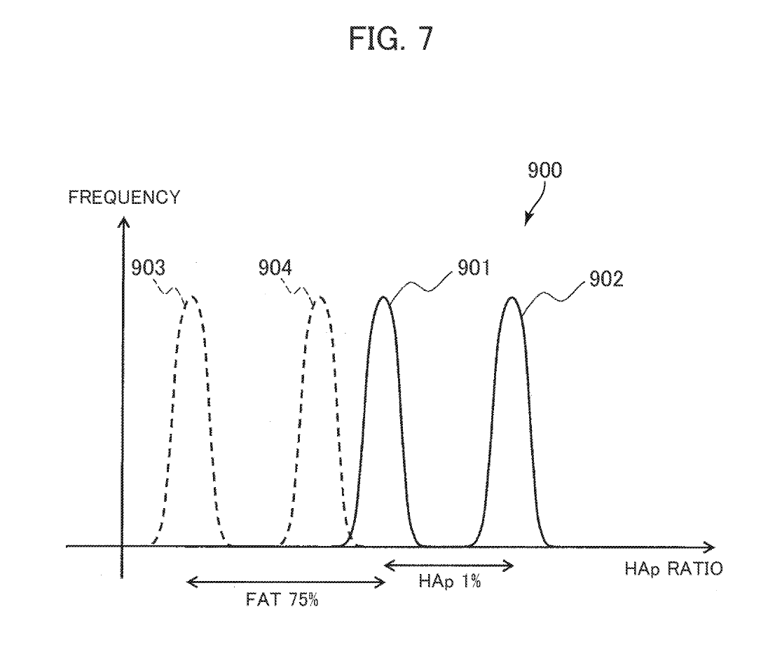

Hereinafter, the explanation about FIG. 4 will be made again. Although it is not easily found from FIG. 5, it can be judged from FIG. 4 that the error distributions of the histograms 701 to 704 (the widths of these histograms) shown in FIG. 5 are derived from the fact that the homogeneous regions 601 to 604 corresponding to the histograms 701 to 704 respectively have elliptical ring structures the direction of each of which is different from the direction of the HAp ratio axis or the direction of the fat ratio axis. Here, the histogram 701 in FIG. 7 corresponds to the homogeneous region 601 in FIG. 6, and the histogram 702 in FIG. 7 corresponds to the homogeneous region 602 in FIG. 6. In addition, the histogram 703 in FIG. 7 corresponds to the homogeneous region 603 in FIG. 6, and the histogram 704 in FIG. 7 corresponds to the homogeneous region 604 in FIG. 6.

Here, it should be noted that, when images respectively based on axes in parallel with the lateral directions of the homogeneous regions 601 to 604 in the scatter plot 600 are considered, images having small statistical errors (images with minimized statistical errors) can be obtained.

The angle processing unit 415 detects the longitudinal direction 611 of any of the homogeneous regions 601 to 604 in the scatter plot 600 (in this case, the longitudinal direction of the homogeneous region 603 is shown), and calculates an angle between the detected longitudinal direction 611 and an arbitrary material axis (in this case, the fat ration axis) as a rotation angle 621. This process is executed at step S122 in FIG. 3. The longitudinal direction 611 shows the correlation direction of the plot points of any of the homogeneous regions 601 to 604, and this direction is calculated using the least-squares method for example.

The angle processing unit 415 rotates the scatter plot 600 in the direction of the calculated rotation angle 621 at step S123 in FIG. 3. Here, the center of the rotation is the origin of the scatter plot 600 in FIG. 4. However, the center of the rotation is not limited to the origin of the scatter plot 600, and it can be any point.

Here, the explanation of a reference sign 631 in FIG. 4 will be made later.

### (Rotated Scatter Plot)

FIG. 6 is a diagram showing an example of a scatter plot after the rotation processing.

The vertical and horizontal axes in FIG. 6 are the same as those in FIG. 4.

In a rotated scatter plot 800, it is found that the longitudinal directions of homogeneous regions 601a to 604a that respectively correspond to the homogeneous regions 601 to 604 in FIG. 4 are perpendicular to the HAp ratio axis. The same is equally true of homogeneous regions other than the homogeneous regions 601a to 604a.

Here, the explanation of a reference sign 821 will be made later.

FIG. 7 is a diagram showing an example of HAp image histograms generated on the basis of the rotated scatter plot.

HAp image histograms 900 shown in FIG. 7 are generated using a technique similar to the technique used for generating the HAp image histograms 700 shown in FIG. 5.

As is clear from a pair of a histogram 901 that shows the error distribution of the homogeneous region 601a in FIG. 6 and a histogram 902 that shows the error distribution of the homogeneous region 602a in FIG. 6, the widths of the error distributions are respectively reduced, that is to say, the error distributions are improved, while the difference between the HAp ratio of the histogram 901 and that of the histogram 902 remains 1%. In other words, the widths of the error distributions of the histogram 901 and the histogram 902 become small respectively. Such histograms as 901 and 902 are obtained as above, which means that an error minimized image obtained by converting the material decomposition image using the rotated scatter plot 800 in FIG. 6 becomes a sharp image. Therefore, the visibility of the image is largely improved, so that it becomes easy to specify the image part in the reconfiguration of the ROIs. A histogram 903 showing the error distribution of the homogeneous region 603a in FIG. 6 and a histogram 904 showing the error distribution of the homogeneous region 604a in FIG. 6 have similar characteristics. The histograms 901 to 904 are histograms having the minimum statistical errors respectively in the meaning that it is assumed that the error distributions of the histograms 901 to 904 are derived from only the HAp ratios. In other words, the “error minimization” in an “error minimized image” means that errors are minimized under the assumption that the relevant error distributions are derived from only the HAp ratios (the ratios of a base material that is a processing target). Incidentally, because the values of the HAp ratios and the values of the fat ratios shown in FIG. 6 and FIG. 7 lose their meanings by the rotation processing, the values of the HAp ratios and the values of the fat ratios shown in FIG. 6 and FIG. 7 serve only as rough indications.

Here, although it seems that there are two kinds of distributions that have the HAp ratios 0% and 1% independently of the fat ratios in the HAp image histograms 700 in FIG. 5, the above two kinds of distributions are divided into four kinds of distributions by two kinds of fat ratios 0% and 75% in the HAp image histograms 900 in FIG. 7. This means that the HAp histograms have lost independency from the fat ratios. Therefore, a material decomposition image before rotation processing becomes more important in a region where fat and HAp are mixed, and it is recommendable that an error minimized image obtained after the rotation processing is secondarily used for an ROI reconfiguration and the like.

Subsequently, the pixel conversion unit 416 replaces the pixels of the material decomposition image obtained as a result of step S111 with the pixels in the scatter plot after the rotation processing (the rotated scatter plot 800), which generates an error minimized image. In other words, the pixel conversion unit 416 generates the error minimized image by replacing the pixels of the material decomposition image having the relation between the HAp ratio and the fat ratio shown in FIG. 4 with the pixels of the material decomposition image having the relation between the HAp ratio and the fat ratio shown in FIG. 6 (S131 in FIG. 3).

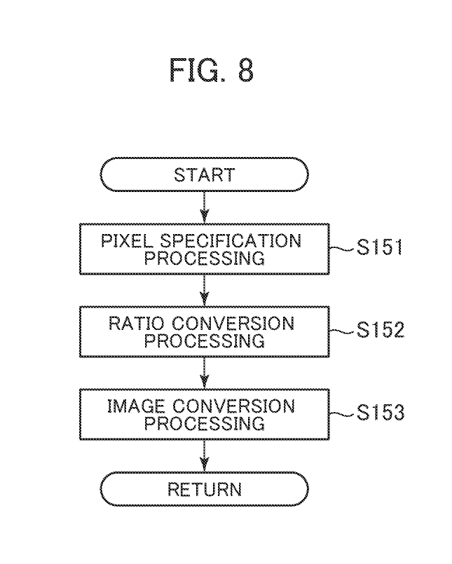

The processing procedure in the pixel conversion unit 416 will be explained with reference to FIG. 8.

FIG. 8 is a flowchart showing the detail procedure of the pixel conversion processing (step S131 in FIG. 3) according to this embodiment.

To put it concretely, the pixel conversion unit 416 performs the following processing.

The pixel conversion unit 416 specifies pixels in the scatter plot 800 in FIG. 6 corresponding to pixels in the scatter plot 600 in FIG. 4 respectively (pixel specification processing: S151). To put it concretely, the pixel conversion unit 416 performs the following processing. For example, a plot point 631 in FIG. 4 and a plot point 821 in FIG. 8 show the same pixel. Therefore, the pixel conversion unit 416 specifies a pixel shown by the plot point 821 in FIG. 8 corresponding to a pixel shown by the plot point 631 in FIG. 4.

Next, the pixel conversion unit 416 converts HAp ratios and fat ratios corresponding to pixels in the scatter plot 600 shown in FIG. 4 into HAp ratios and fat ratios corresponding to pixels in the scatter plot 800 shown in FIG. 6 (ratio conversion processing: S152). For example, the pixel conversion unit 416 converts the values of a HAp ratio and a fat ratio shown by the plot point 631 (a pixel) in FIG. 4 into the values of a HAp ratio and a fat ration shown by the plot point 821 (a pixel) in FIG. 8.

The pixel conversion unit 416 converts the pixels of a material decomposition image in accordance with this conversion method (image conversion processing: step S153). As a result, an error minimized image is generated.

The entirety of M images (M=2 in this embodiment) obtained as a result of the processing performed by the pixel conversion unit 416 is referred to as a rotated image. Incidentally, M is equal to the number of the base materials. As described above, a rotated image includes M images (M=2 in this embodiment), and one of the M images is an error minimized image (corresponding to HAp in this embodiment). Furthermore, other images are images that have larger statistical errors (corresponding to fat in this embodiment). In this embodiment, an image with its statistical errors minimized (corresponding to HAp in this embodiment) among the obtained M images (M=2 in this embodiment) is an error minimized image.

Because pixel values in the error minimized image have lost the meanings of original HAp ratios, it will be useful if the pixel values in the error minimized image are converted into values compliant with Hounsfield values used for typical CT. In this case, the assumed CT values of base materials (for example, +60 for blood, and −70 for fat) can be used as standard values.

Here, it is not always indispensable to display the scatter plot 600 shown in FIG. 4 and the rotated scatter plot 800 shown in FIG. 6 on the display device 406. However, for the purpose of checking whether the homogeneous regions 601 to 604 are parallel with each other both in the longitudinal directions 611 (FIG. 4) and in the lateral direction of the homogeneous regions 601 to 604, and for the purpose of adjusting a rotation angle fine, it is useful to display the scatter plot 600 shown in FIG. 4 and the rotated scatter plot 800 shown in FIG. 6 on the display device 406.

In addition, although the angle processing unit 415 calculates a rotation angle, and the angle processing unit 415 performs rotation processing in accordance with the rotation angle, it is conceivable that a user manually rotates the scatter plot 600 using an input device 405 (FIG. 2) such as a mouse.

Rotation processing performed by the angle processing unit 415 has an advantage that the result of rotating an image can be checked as well, and manual rotation processing performed by a user has an advantage that calculation cost for the manual rotation is small and the result of the manual rotation can be displayed at once.

Furthermore, in the case where, after rotation processing is performed, error minimized images for the regions of the test substance A1 (FIG. 1) are slightly different from each other owing to the imperfection of beam hardening correction, it is conceivable that rotation processing is performed for each of the regions.

### (Operation Screen)

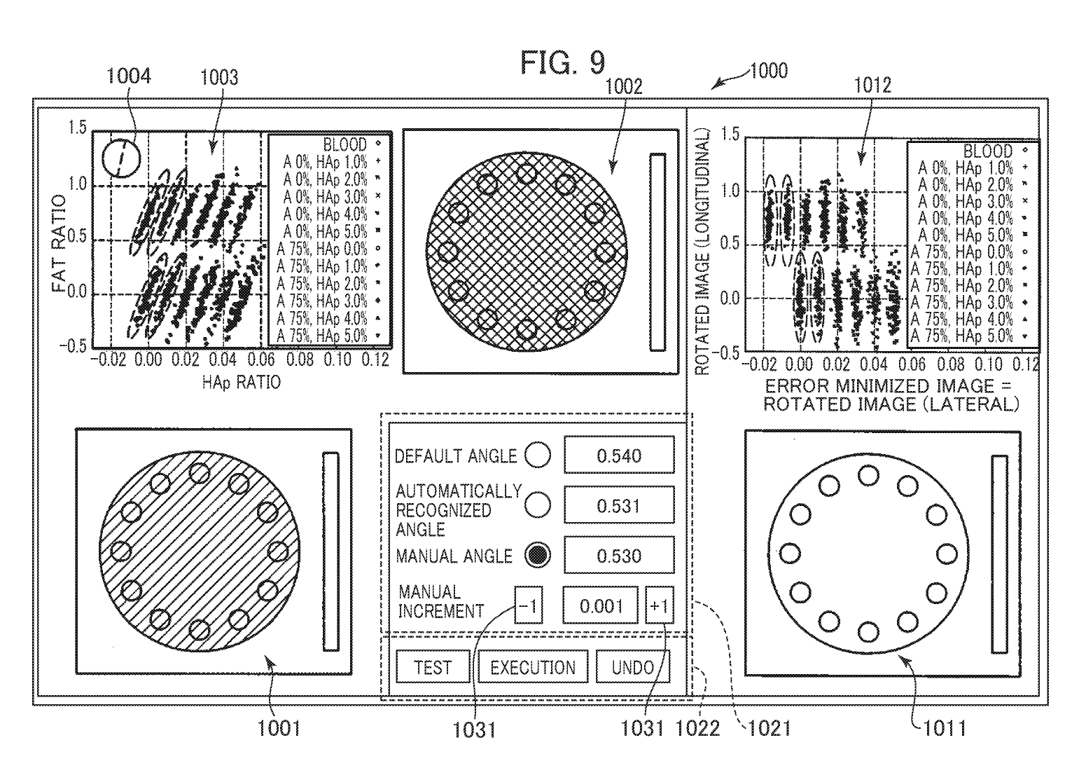

FIG. 9 is a diagram showing an example of an operation screen according to this embodiment.

Here, it will be assumed that typical operations in the related art such as the photographing processing (step S101 in FIG. 3) and the image reconfiguration processing (step S103 in FIG. 3) are performed using a screen other than the operation screen 1000 shown in FIG. 9.

In the operation screen 1000, a first material decomposition image area 1001 is an area on which a material decomposition image (HAp image), which is an input image, is displayed. In addition, a second material decomposition image area 1002 is an area on which a material decomposition image (fat image), which is an input image, is displayed. As above, as many material decomposition image areas as base materials are displayed.

Furthermore, a scatter plot area 1003 is an area on which a scatter plot generated at step S121 in FIG. 3 is displayed. Additionally, a marker 1004 showing the longitudinal direction of a homogeneous region is displayed in the scatter plot displayed on the scatter plot area 1003.

In addition, an error minimized image area 1011 is an area on which an error minimized image after rotation processing is displayed. Furthermore, a scatter plot after rotation processing is displayed on a rotated scatter plot area 1012.

In addition, a user can select and adjust a rotation angle using a rotation angle operation unit 1021. Examples of the choices of the rotation angle are “Default Angle”, “Automatically Recognized Angle”, “Manual Angle”, and “Manual Increment”.

“Default Angle” is the precalculated value of an angle that is determined by the base material of a material decomposition image and the setting condition of energy windows and independent of the phantom. In other words, “Default Angle” is a rotation angle that is predetermined.

Furthermore, ideally speaking, statistical errors are corrected by beam hardening correction so as to be substantially small with being little affected by beam hardening. With this, the rotation angle is set independently of the shape and size of the test substance A1 (FIG. 1).

However, there are some cases where uncorrected/overcorrected components remain after the beam hardening correction. In such cases, the rotation angle is affected by the test substance A1.

If “Automatically Recognized Angle” is selected, the angle processing unit 415 automatically recognizes a region whose homogeneity is high (homogeneous region) in the scatter plot 600 (FIG. 4), and automatically detects the rotation angle from the homogeneous region. In other words, “Automatically Recognized Angle” is an angle calculated by the angle processing unit 415.

In addition, as for an RIO, a user can designate the ROI regarding an error minimized image displayed on the error minimized image area 1011. In other words, although, in the abovementioned setting method of an ROI, the scatter plot generation processing unit 414 judges and sets an ROI, a user can also set an ROI.

Furthermore, because it is preferable that an ROI is set regarding an image with small statistic errors, a feedback loop in which an ROI is updated from an already-obtained error minimized image is considered to be useful. “A feedback loop in which an ROI is updated from an already-obtained error minimized image” means the following. First, a user once sets ROIs regarding material decomposition images before rotation processing (regarding the images displayed in the first material decomposition image area 1001 and in the second material decomposition image area 1002). Next, after rotation processing, the output processing unit 417 displays the ROIs set regarding the material decomposition image before the rotation processing on the error minimized image displayed on the error minimized image area 1011, and makes the user judge whether the reconfiguration of the ROIs is necessary or not.

“Manual Angle” is used when a user inputs an arbitrary angle as a rotation angle, and for example, it is used for a special purpose. The value of the rotation angle can be directly edited via the input device 405, or it can be finely adjusted by giving editable values +1° or −1° to the currently set value of the rotation angle as a “Manual Increment” using buttons 1031 each of which corresponds to +1° or −1°.

Alternatively, when a scatter plot is rotated using the mouse, the rotation angle of the scatter plot reflecting the mouse operation can be displayed on the operation screen 1000 as a “Manual Angle”.

Incidentally, a rotation angle given from the rotation angle operation unit 1021 is reflected in the marker 1004, and a user can check the set rotation angle by visually perceiving the marker 1004.

As shown in FIG. 9, any choice of “Default Angle”, “Automatically Recognized Angle”, and “Manual Angle” can be designated by pushing a radio button corresponding to a choice to be executed.

The execution operation unit 1022 is an interface for performing the rotation processing. The execution operation unit 1022 includes some buttons such as a test button using which only a scatter plot is rotated on a trial basis, an execution button which is used for making the rotation processing reach the generation of an error minimized image, and an undo button which cancels the content of the previous execution.

It is also conceivable that the abovementioned “Manual Increment” includes a function using which only a scatter plot is rotated on a trial basis. In other words, the rotation of a scatter plot executed by information input from “Manual Increment” is a rotation executed on a trial basis, and it is conceivable that, in order to generate an error minimized image on the basis of this rotation, the execution button has to be pushed. Meanwhile, it is also conceivable that a function that information input from “Manual Increment” is instantaneously reflected in a scatter plot after a rotation (displayed in the rotated scatter plot area 1012) is added to the choice of “Manual Increment”. Here, the rotation angle calculation processing (step S122) includes a broad processing concept including angle processing examples performed by “Default Angle”, “Automatically Recognized Angle”, and “Manual Angle”.

According to the technology disclosed in Patent Literature 1, a small-statistical-error image, whose errors are equal to the errors of an image obtained by CT using a current mode detector or smaller, is obtained. In other words, a sharp line attenuation coefficient image can be obtained. However, in the case of the technology disclosed in Patent Literature 1, although small statistical errors are obtained, it is not ensured that the small statistical errors are minimized errors. In other words, the technology disclosed in Patent Literature 1 cannot show to what extent statistical errors are improved. In addition, the technology disclosed in Patent Literature 1 necessitates additional image reconfiguration processing in order to obtain a line attenuation coefficient image using weighted addition. In other words, in the technology disclosed in Patent Literature 1, the image reconfiguration processing has to be performed twice. Judging from the fact that a successive approximation-type image reconfiguration, which has been recently used widely, requires a high calculation cost, since the additional image reconfiguration processing is necessary for the technology disclosed in Patent Literature 1, the technology requires a high calculation cost.

On the other hand, the image generation apparatus 400 according to this embodiment does not require additional image reconfiguration processing, and can minimize errors using only rotation processing that requires a low calculation cost. Furthermore, in this embodiment, it is ensured that errors are minimized.

Here, although the number of base materials M has been set to 2 so far in this embodiment, the number of base materials M may be set to 3. In this case, a scatter plot becomes three-dimensional. A three-dimensional rotation is a two-degree-of-freedom operation, and usually the three-dimensional rotation is specified by two values, that is to say, by the value of a polar angle and the value of an azimuth angle. In other words, when the number of base material is 3, the shape of a homogeneous region in a scatter plot becomes an approximate ellipsoid. A homogeneous region whose shape is an approximate ellipsoid has two longitudinal directions that are perpendicular to each other, and one lateral direction that is perpendicular to the two longitudinal directions.

The angle processing unit 415 calculates a rotation angle that makes the lateral direction of a homogeneous region parallel with an axis corresponding to a processing target base material in a scatter plot (three-dimensional), and rotates the scatter plot with this rotation angle. With such an operation, an error minimized image can be obtained as is the case with the number of base materials M=2.

Furthermore, if the calculation of the rotation angle of a homogeneous region is executed on the basis of the longitudinal direction of the homogeneous region, it can be more easily executed. However, in the case where the lateral direction of the homogeneous region can be directly known and only an error minimized image is a target of interest among rotated images, it is conceivable that the angle processing unit 415 makes this lateral direction parallel with an axis corresponding to a processing target base material. In this case, although the two values of a polar angle and an azimuth angle are needed for a rotation, the degree of freedom of the rotation is 1, and therefore an arbitrary pair of a polar angle and an azimuth angle can be adopted among a number of pairs of a polar angle and an azimuth angle. In the case of M=2, a rotation angle can be calculated similarly on the basis of the lateral direction of a homogeneous region.

Even in the case of M≧4, the above calculation method can be used expansively as well.

According to this embodiment, an error minimized image having minimized statistical errors can be obtained at a low calculation cost.

In addition, an error minimized image having minimized statistical errors can be obtained by replacing the pixels of a material decomposition image with the pixels of the relevant scatter plot 800 (FIG. 6) on which rotation processing has been performed without performing image reconfiguration processing on the relevant count projection data. With this, an image having minimized statistical errors can be obtained at a low calculation cost.

Furthermore, the image generation apparatus 400 according to this embodiment can obtain an image having small statistical errors regarding a material decomposition image from which energy information is obtained but whose statistical errors are large.

Although the scatter plot 600 shown in FIG. 4 is rotated so that the gradients of the longitudinal directions 611 of the homogeneous regions 601 to 604 (FIG. 4) become perpendicular to the HAp ratio axis shown in FIG. 6 in this embodiment, the scatter plot 600 may be rotated so that the gradients become perpendicular to the fat ratio axis.

In addition, although this embodiment is applied to the X-ray CT apparatus 100, this embodiment can also be applied to various medical diagnostic imaging apparatuses that utilize PET (Positron Emission Tomography), MRI, PET-CT, or the like. Furthermore, although this embodiment has been described so far on the assumption that the X-ray CT apparatus 100 includes a pulse mode X-ray detector as the X-ray detector 321, a dual energy CT apparatus including a current mode X-ray detector 321 can be used without limiting to including the pulse mode X-ray detector. If the dual energy CT apparatus is used, a method in which an X ray having two or more kinds of spectra is irradiated from the X-ray tube 311, a method in which the X-ray detector 321 detects information regarding different energy distributions, and the like can be adopted. Moreover, in this embodiment, image reconfiguration processing is performed using count projection data acquired from the X-ray CT apparatus 100. However, it is conceivable that, count projection data is stored in a database in advance, and image reconfiguration processing is performed using count projection data stored in this database. The abovementioned method can be used in both cases where a pulse mode X-ray detector is used as the X-ray detector 321 and where a current mode X-ray detector 321 is used.

The present invention is not limited to the above embodiment, and various modification examples can be included. For example, the above embodiment has been described in detail in order to make the present invention easy to understand, and therefore all the components described so far are not always indispensable for the present invention. In addition, this embodiment can be changed by adding a different configuration to a part of the configuration of this embodiment, by deleting a part of the configuration of this embodiment, or by replacing a part of the configuration of this embodiment with a different configuration.

Furthermore, it is conceivable that some or all of each of the above-described configurations, functions, units 411 to 417, a storage device 403, and the like are realized by hardware, for example, through designing with use of integrated circuits. Alternatively, as shown in FIG. 2, it is also conceivable that the above-described configurations, functions, and the like are realized by software through the operations of processors such as the CPU in which the processors interpret programs, which realize the functions of the above-described configurations and the like, and executes the programs. Information regarding the programs, tables, files, and the like that are used for realizing various functions can be stored on storage devices such as memories and SSDs (Solid State Drives) or on storage media such as IC (Integrated Circuit) cards, SD (Secure Digital) cards, and DVDs (Digital Versatile Discs) as well as on HDs (hard discs).

In addition, in the embodiment, control lines and information lines are shown in the case where they are indispensable for explaining each embodiment, therefore all control lines and information lines necessary for realizing each embodiment as a product are not shown. It is conceivable that in reality almost all components in almost every embodiment are interconnected.

### LIST OF REFERENCE SIGNS

100: X-ray CT Apparatus
200: Input Apparatus
300: Photographing Apparatus
400: Image Generation Apparatus
406: Display Device (Display Unit)
410: Processing Unit
411: Data Acquisition Unit
412: Image Reconfiguration Processing Unit
413: Base Material Decomposition Processing Unit
414: Scatter Plot Generation Unit
415: Angle Processing Unit (Error Minimizing Unit)
416: Pixel Conversion Unit (Conversion Unit)
600, 800: Scatter Plot
601 to 604, 601a to 604a: Homogeneous Region
700, 900: HAp Image Histograms
701 to 704, 901: Histogram
1000: Operation Screen
1001: First Material Decomposition Image Area
1002: Second Material Decomposition Image Area
1003: Scatter Plot Area
1004: Marker
1011: Error Minimized Image Area
1021: Rotation Angle Operation Unit
1022: Execution Operation Unit

1. An image generation apparatus comprising:
a scatter plot generation unit that generates a scatter plot in which the axes represent the concentrations of base materials used in base material decomposition and pixels of a material decomposition image output as a result of the base material decomposition are plotted against the corresponding concentrations of the base materials of the material decomposition;
an error minimizing unit that rotates the scatter plot in a direction that minimizes the statistical errors of plot points plotted on the scatter plot; and
a conversion unit that converts the material decomposition image on the basis of the pixels in the scatter plot rotated by the error minimizing unit.

2. The image generation apparatus according to claim 1,
wherein the error minimizing unit calculates the gradient of the correlation direction of the plot points on the scatter plot and minimizes the statistical errors of the plot points plotted on the scatter plot by rotating the scatter plot so that the calculated gradient of the correlation direction becomes perpendicular to an axis corresponding to a processing target.

3. The image generation apparatus according to claim 1,
wherein the conversion unit converts the material decomposition image on the basis of the pixels in the scatter plot the statistical errors on which are minimized by replacing information about the pixels of the material decomposition image with information about the pixels in the rotated scatter plot.

4. The image generation apparatus according to claim 1, further comprising an output processing unit that displays at least the scatter plot before the abovementioned rotation and the scatter plot after the abovementioned rotation on a display unit.

5. The image generation apparatus according to claim 1,
wherein the base material decomposition is executed on a line attenuation image obtained from an X-ray CT including a pulse mode X-ray detector.

6. An image generation method,
wherein an image generation apparatus that converts a material decomposition image:
generates a scatter plot in which the axes represent the concentrations of base materials used in base material decomposition and pixels of a material decomposition image output as a result of the base material decomposition are plotted against the corresponding concentrations of the base materials of the material decomposition;
rotates the scatter plot in a direction that minimizes the statistical errors of plot points plotted on the scatter plot; and
converts the material decomposition image on the basis of the pixels in the rotated scatter plot.

7. The image generation method according to claim 6,
wherein the image generation apparatus:
calculates the gradient of the correlation direction of the plot points on the scatter plot; and minimizes the statistical errors of the plot points plotted on the scatter plot by rotating the scatter plot so that the calculated gradient of the correlation direction becomes perpendicular to an axis corresponding to a processing target.

8. The image generation method according to claim 6,
wherein the image generation apparatus converts the material decomposition image on the basis of the pixels in the scatter plot the statistical errors on which are minimized by replacing information about the pixels of the material decomposition image with information about the pixels in the rotated scatter plot.

9. The image generation method according to claim 6,
wherein the image generation apparatus displays at least the scatter plot before the abovementioned rotation and the scatter plot after the abovementioned rotation on a display unit.

10. The image generation method according to claim 6,
wherein the base material decomposition is executed on a line attenuation image obtained from an X-ray CT including a pulse mode X-ray detector.

11. An X-ray CT apparatus comprising:
a scatter plot generation unit that generates a scatter plot in which the axes represent the concentrations of base materials used in base material decomposition and pixels of a material decomposition image output as a result of the base material decomposition are plotted against the corresponding concentrations of the base materials of the material decomposition;
an error minimizing unit that rotates the scatter plot in a direction that minimizes the statistical errors of plot points plotted on the scatter plot; and
a conversion unit that converts the material decomposition image on the basis of the pixels in the scatter plot rotated by the error minimizing unit.

12. The X-ray CT apparatus according to claim 11,
wherein the error minimizing unit calculates the gradient of the correlation direction of the plot points on the scatter plot and minimizes the statistical errors of the plot points plotted on the scatter plot by rotating the scatter plot so that the calculated gradient of the correlation direction becomes perpendicular to an axis corresponding to a processing target.

13. The X-ray CT apparatus according to claim 11,
wherein the conversion unit converts the material decomposition image on the basis of the pixels in the scatter plot the statistical errors on which are minimized by replacing information about the pixels of the material decomposition image with information about the pixels in the rotated scatter plot.

14. The X-ray CT apparatus according to claim 11,
wherein the base material decomposition is executed on a line attenuation image obtained from an X-ray CT including a pulse mode X-ray detector.
